(19) United States
(12) Patent Application Publication
(10) Pub. No.: US 2025/0019726 A1
(43) Pub. Date: Jan. 16, 2025

(54) A PROCESS TO TREAT A CARBON DIOXIDE COMPRISING GAS

(71) Applicant: PAQELL B.V., Utrecht (NL)

(72) Inventors: Dandan LIU, Wageningen (NL); Frederikus DE RINK, Amersfoort (NL); Johannes Bernardus Maria KLOK, Rhenen (NL)

(73) Assignee: PAQELL B.V., Utrecht (NL)

(21) Appl. No.: 18/712,073

(22) PCT Filed: Nov. 24, 2022
(86) PCT No.: PCT/EP2022/083088

§ 371 Date: May 21, 2024

(30) Foreign Application Priority Data
Nov. 26, 2021 (NL) 2029926

(51) Int. Cl. C12P 5/02; C25B 3/03; C25B 9/40; C25B 11/037; C25B 11/063; C25B 11/081

(52) U.S. Cl. CPC C12P 5/023; C25B 3/03; C25B 9/47; C25B 11/037; C25B 11/063; C25B 11/081

(57) ABSTRACT

The invention is directed to a process to convert carbon dioxide to methane by contacting an aqueous solution comprising dissolved carbon dioxide with an electron charged packed bed comprising of a carrier and a biofilm of microorganisms under anaerobic conditions wherein the pH of the aqueous solution is above 7.5 and wherein the aqueous solution comprises between 0.3 and 4 M sodium cations or between 0.3 and 4 M sodium and potassium cations and more than 20 mM phosphate ions.

The invention is directed to a process to treat a carbon dioxide comprising gas wherein carbon dioxide is converted to methane in the presence of an electron charged packed bed comprising of a carrier and microorganisms under anaerobic conditions.

CN106947688 describes a system and process for reducing carbon dioxide to produce methane through microorganisms/photoelectric-coupling. The system includes an anode chamber and a cathode chamber. In the cathode chamber microorganisms are present in a buffered solution where the KH₂PO₄ and Na₂HPO₄ achieve the buffering effect. The disclosed total salt concentration is low resulting in that non-halophilic microorganisms are expected to be present.

CN112376073 describes a system and process for reducing carbon dioxide to produce methane in a bioelectrochemical system provided with an anode chamber and a cathode chamber. In the cathode chamber microorganisms are present in a buffered solution where the KH₂PO₄ and Na₂HPO₄ achieve the buffering effect. The disclosed total salt concentration is low resulting in that non-halophilic microorganisms are expected to be present.

A journal article titled Granular Carbon-Based Electrodes as Cathodes in Methane-Producing Bioelectrochemical Systems, Dandan Liu, Marta Roca-Puigros, Florian Geppert, Leire Caizán-Juanarena, Susakul P. Na Ayudthaya, Cees Buisman and Annemiek ter Heijne, Frontiers in Bioengineering and Biotechnology, June 2018|Volume 6, article 78 described a process where carbon dioxide is converted to methane in the presence of an electron charged packed bed comprising of activated carbon granules and a mixed culture microorganisms under anaerobic conditions. The CO₂ was supplied as a gas to an aqueous solution having a pH of 6.5. The disclosed total salt concentration is low resulting in that non-halophilic microorganisms are expected to be present. The biocathode consisting of the electron charged packed bed comprising of activated carbon granules and a mixed culture microorganisms was charged for 2 minutes alternating with no charging for 4 minutes. The reported “current to methane” efficiency was 55%. The reported overall energy efficiency was 25%.

NL2026669 describes an improved process with respect to the above referred Journal article of Dandan Lui et al. In this process carbon dioxide is converted to methane by contacting an aqueous solution comprising dissolved carbon dioxide with an electron charged packed bed comprising of a carrier and a biofilm of halophile microorganisms under anaerobic conditions, wherein the dissolved carbon dioxide is present for more than 90 mol % as a bicarbonate ion and/or as a carbonate ion. It has been found that the energy efficiency is substantially improved to values of 40% at these more alkaline conditions. A problem of this process is that the higher energy efficiency drops after a certain time when the process is performed for a prolonged period of time.

It is an object of the present invention to improve the stability of the process disclosed in NL2026669.

This object is achieved by the following process. A process to convert carbon dioxide to methane by contacting an aqueous solution comprising dissolved carbon dioxide with an electron charged packed bed comprising of a carrier and a biofilm of microorganisms under anaerobic conditions, wherein the pH of the aqueous solution is above 7.5, wherein the aqueous solution comprises between 0.3 and 4 M sodium cations or between 0.3 and 4 M sodium and potassium cations and wherein the aqueous solution comprises more than 20 mM phosphate ions.

Applicants found that when the process is performed in the presence of more than 20 mM phosphate ions a more stable process is obtained wherein the energy efficiency improves to values of around 60%.

The dissolved carbon dioxide may be present as aqueous carbon dioxide, carbonic acid, bicarbonate ions and as carbonate ions. A major part of the dissolved carbon dioxide in the aqueous solution is present as a bicarbonate ion and/or as a carbonate ion. Preferably more than 90 mol % and even more preferably more than 95 mol % of the dissolved carbon dioxide in the aqueous solution is present as a bicarbonate ion and/or as a carbonate ion. The pH conditions at which these compounds are present in an aqueous solution is above 7.5, preferably above 7.7 and more preferably above 8 and even more preferably in the range of from 8 to 10 and most preferably of from 8.5 to 9.5. These alkaline conditions may be achieved by a basic salt formed between a weak acid and a strong base, such as sodium bicarbonate and potassium bicarbonate. Such basic salt may be formed by adding sodium cations or sodium and potassium cations. The aqueous solution comprises between 0.3 and 4 M sodium cations or between 0.3 and 4 M sodium and potassium cations. Preferably the aqueous solution comprises between 0.4 and 2 M and even more preferably between 0.5 and 1.5 M sodium cations or sodium and potassium cations. The resulting aqueous solution having a high salt concentration is a buffered solution further comprising sodium carbonate and sodium bicarbonate or potassium carbonate and potassium bicarbonate or their mixtures.

Only a small content of more than 20 mM, preferably more than 40 mM and even more preferably more than 50 mM of phosphate ions is required. It is suggested that the phosphate ions suppress microbial growth of competing microorganisms which consume electrons and form other products. For this reason only small contents are required. The upper limit may be the saturation concentration will be determined by factors like scaling, which is suitably to be avoided. Contents of up to and even above 0.5 M are conceivable. For practical reasons one would operate the process at low phosphate ion contents. The phosphate ions may be added to the aqueous solution as a salt and preferably as an alkaline salt like sodium phosphate or potassium phosphate. The latter are preferred because sodium and optionally potassium ions are according to the invention present in the aqueous solution.

The aqueous alkaline solution suitably further comprises nutrients for the microorganisms. Examples of suitable nutrients are nutrients such as ammonium, vitamin and mineral elements as may be present as part of a so-called Wolfe's mineral solution. It may be desired to add such nutrients to the aqueous alkaline solution in order to maintain active microorganisms.

The anaerobic conditions are suitably achieved by performing the process in the absence of molecular oxygen, preferably also in the absence of other oxidants such as for example nitrate. By ‘in the absence of molecular oxygen’ is meant that the concentration of molecular oxygen in the loaded aqueous solution in this process is at most 10 UM molecular oxygen, preferably at most 1 μM, more preferably at most 0.1 μM molecular oxygen. Sulfate, which may be regarded to be an oxidant, may be present at low concentrations of for example 160 UM, as part of the earlier referred to Wolfe's mineral solution. It has been found that the sulfate at these low concentrations does not negatively influence the desired conversion of carbon dioxide.

The process is performed by contacting the aqueous solution with an electron charged packed bed comprising of activated carbon granules and microorganisms under anaerobic conditions wherein carbon dioxide is converted to methane. The microorganisms may be a mixed culture of microorganisms or a monoculture. The mixed culture of microorganisms is suitably obtained from an anaerobically grown culture. Suitably the mixed culture comprises hydrogenotrophic methanogens, such as for example Methanobacterium. Further microorganisms may be present, including anaerobic or facultative anaerobic bacteria, for example Proteobacteria, such as for example Deltaproteobacteria and Betaproteobacteria.

At the high salt concentration conditions of the process of this invention halophile microorganisms will dominate the culture even when the starting culture is obtained from an anaerobically grown culture which consisted of mainly non-halophile microorganisms. Examples of halophile microorganisms which may be present in the process are slight halophiles and moderate halophiles belonging to genus level of Bathyarchaeia, Methanobacterium, Methanosaeta, Candidatus Methanogranum, Marinobacter, Balneolaceae, Desulfovibrionaceae, Acetobacterium, Acidaminococcaceae, Halothiobacillus, Spirochaetaceae, Paludibacter, Rhodobacteraceae, Desulfobacteraceae, Desulfuromonadaceae, Geobacteraceae, Solimonadaceae, Halomonadaceae, Vibrionaceae, Ectothiorhodospiraceae, Oceanospirillaceae, Lentimicrobiaceae and/or Synergistaceae.

The mixed culture microorganisms is preferably obtained from an anaerobic system, such as an anaerobically grown culture. The mixed culture may therefore be obtained from the sludge of an anaerobic bioreactor, such as an anaerobic fermenter, for example one used for anaerobic chain elongation; an anaerobic digestion reactor, for example an upflow anaerobic sludge blanket reactor (UASB); Other suitable bioreactors for providing the sludge are expended granular sludge bed (EGSB), a sequential batch reactor (SBR), a continuously stirred tank reactor (CSTR) or an anaerobic membrane bioreactor (AnMBR). In the present context, the term sludge refers to the semi-solid flocs or granules containing a mixed culture of microorganisms.

The carrier may be any carrier which provides a surface for the biofilm and has a sufficient capacitance property. The preferred carrier is biocompatible and has a 3D granular structure for attachment of the microorganisms and to enhance the mass transfer of the bulk solution and the electrode. Preferably the carrier is carbon based. Preferably the carrier is comprised of activated carbon granules. Suitably the electrodes of the cathode are modified by activated carbon.

The packed bed of the carrier suitably comprises of granules of activated carbon for example, activated biochar. Suitably the bed is a packed bed of activated carbon granules or activated biochar granules having an activated surface area of between 500 and 1500 m²/g and wherein the microorganisms are present as a biofilm on the surface of the activated surface area. The high surface area provides a surface on which the microorganisms are present. Part of the microorganisms may be present planktonically. A high surface area per volume thus provides a higher capacity to perform the desired reaction of carbon dioxide to methane per volume of reactor space.

The dimensions of the granules are suitably such that on the one hand a mass transport of the aqueous fractions is possible in the spaces between the granules without causing a high pressure drop. This means that there will be a practical lower limit with respect to the dimensions of the granules. On the other hand the granules should not be too large because this would result in long travel distances within the micropores of the activated carbon granules. The volume based diameter of the granules may be between 0.5 and 10 mm and preferably between 1 and 4 mm.

The electron charged packed bed comprising of activated carbon granules is preferably part of a biocathode in a bioelectrochemical system further comprising an anode. The biocathode suitably comprises a volume of activated carbon granules arranged in a packed bed. The packed bed contacts with a current collector, which may be a surface of a conductive electrode material, such as a carbon comprising materials such as a graphite plate or felt or a metal mesh, preferably a stainless-steel mesh. The current collector is arranged such that the packed bed may be charged with electrons from said current collector.

The packed bed will further be positioned in a cathode space of the bioelectrochemical system which is fluidly connected to an anode space of the bioelectrochemical system and separated from said anode space by an ion exchange membrane, preferably a cation exchange membrane. In order to compact the packed bed of activated carbon granules it may be preferred to add inert particles, like glass beads, to the anode space such to counterbalance the pressure exercised by the packed bed on the ion exchange membrane.

The aqueous solution as present at the anode is referred to as the anolyte and the aqueous solution as present at the cathode is referred to as the catholyte. Suitably a recirculation is performed where part of the catholyte is fed to the anode to become part of the anolyte and part of the anolyte is fed to the cathode to become part of the catholyte. It is found that when such a recirculation is performed a more efficient process is obtained wherein the major part of the dissolved carbon dioxide in the aqueous solution is present as a bicarbonate ion and/or as a carbonate ion.

Preferably the content of oxygen as may be present in the anolyte should be low when this is fed to the cathode to become part of the catholyte. The oxygen content may be decreased by removing oxygen from this anolyte stream by means of a gas-liquid separation or a degassing unit. Alternatively physical or chemical oxygen scavengers such as sulfite or an organic scavenger may be used to lower the oxygen content. Also the anolyte may be purged with O₂ free gasses, such as N₂ and/or CO₂. Oxygen may also be removed from the anolyte by electrochemical removal techniques.

Preferably the content of methane as may be present in the catholyte should be low when this is fed to the anode to become part of the anolyte. The methane content may be decreased by removing methane from this anolyte stream by means of a gas-liquid separation.

The packed bed of activated carbon granules may be charged in such a system by applying a potential to the bioelectrochemical system resulting in a current between biocathode and anode such that electrons are donated at the anode and at the cathode electrons are supplied to the packed bed. At the anode an oxidation reaction, such as water oxidation, takes place providing the required electrons. The potential may be achieved by an external power supply generating electricity, like for example power generated by wind and/or solar. Alternatively the electrons and thus the power supply may be partially donated by a chemical reaction at the anode. An example of such a chemical reaction is the organic matter (i.e. COD) oxidation as described in Cerrillo, M., Viñas, M. and Bonmati, A. (2017) Unravelling the active microbial community in a thermophilic anaerobic digester-microbial electrolysis cell coupled system under different conditions. Water Research 110, 192-201.

The anode will be placed in the anode space and may be made of a material suited for the oxidation of the chosen electron donor. Preferred materials for water as the electron donor are platinum, ruthenium, titanium, tantalum coated with iridium and their mixtures. An example of a suitable anode material is a iridium-tantalum-coated titanium plate. Preferably the anode is a ruthenium coated titanium mesh. It has been found that the electrochemically catalytic property for water oxidation of the iridium-tantalum coated titanium mesh is higher than the platinum-iridium-coated titanium anode. The experimental results have shown that the required anode potential for water splitting is: 1.14 V vs. Ag/AgCl (3 M KCl) at a current density of 5 A/m². This is much lower than the 1.9 V vs. Ag/AgCl (3 M KCl) at the same current density observed in previous experiments with the platinum-iridium-coated titanium anode material. A lower anode potential requires less energy input. When the current density was increased to 10 A/m², it was expected that the anode potential would increase. However, the actual increase of the anode potential was negligible.

The charged packed bed is suitably charged to a capacitance of between 10 to 100 F/g. Preferably charging is performed in a bioelectrochemical system comprising a biocathode, an anode and a cation exchange membrane. The electron charged packed bed is part of the biocathode. The packed bed is charged by applying a voltage/current to the bioelectrochemical system resulting in a current between biocathode and anode for a certain time resulting in that the packed bed is loaded with electrons. Preferably the packed bed is charged by applying a current density to the cathode electrode of between 2 and 200 A/m² and preferably between 2.5 and 120 A/m²; or by applying a cathode potential to the current collector of the biocathode which is less negative than the hydrogen evolution potential. The cathode potential at which hydrogen evolution occurs is depending on the pH at which the process is operated. Preferably, as the pH ranges typically between 7.5 and 9.0, the range of the cathode potential varies between −0.50 and −0.74V vs. Ag/AgCl (3 M KCl).

The electron charged packed bed does not necessarily have to be connected to an external power supply such that no power is supplied when performing the process. When the packed bed is sufficiently charged with electrons it is found that the process performs for a prolonged period of time. For example the process may be performed for between 0.03 and 12 hours, preferably between 0.05 and 10 hours, in a situation wherein no power is supplied to the electron charged packed bed. This is advantageous because this allows the use of a non-continuous power supply generating electricity, preferably a sustainable and renewable external power supply, such as for example solar and/or wind. The capability of the process to operate when such a non-continuous power supply is temporally not available is advantageous.

The process may be performed using an electron charged packed bed as part of the above described bioelectrochemical system wherein no power is supplied to the electron charged packed bed of the bioelectrochemical system. In such an embodiment the packed bed is charged before performing the process as described above by applying a potential to the bioelectrochemical system resulting in a current between biocathode and anode. The process may also be performed when the packed bed is charged as described above. Also possible is that the process is performed wherein the packed bed is alternatingly charged and not charged because of the absence of an external power supply. In this embodiment some net charging will take place when performing the process. The system will then be connected to an external power supply to supply power.

The process may also be performed in more than one bioelectrochemical system, each system comprising of the biocathode and an anode, and wherein one bio electrochemical system performs the process and another bio electrochemical system is charged. The system performing the process may be performed while no power is supplied to the electron charged packed bed. To the bio electrochemical system which is charged power is supplied such that the packed bed is charged with electrons. Optionally a further bioelectrochemical system of the more than one bioelectrochemical system performs the process while the packed bed is charged by applying a potential/current to the bioelectrochemical system.

Suitably the packed bed comprising of carrier and a biofilm of microorganisms is obtained in an activation step. The activation step is performed at the pH ranges described above for the process steps (i) and (ii) and under anaerobic conditions and by supplying a current at a cathode potential which is more positive than the theoretical hydrogen evolution potential at −0.71 V vs Ag/AgCl (3 M KCl) at pH of 8.5 to the packed bed comprising of carrier and a biofilm of microorganisms from a sludge of an anaerobic wastewater treatment plant. The theoretical hydrogen evolution potential is pH dependent. For example, at a pH of 7 the theoretical hydrogen evolution potential is-0.61 V vs Ag/AgCl (3 M KCl). It has been found that the resulting packed bed, especially comprising of activated carbon granules or activated biochar and a mixed culture microorganisms, is more robust and avoids hydrogen evolution at the cathode when compared to when such an activation does not take place. The activation is preferably performed until stable and optimal potential is obtained after turning on the current supply.

The process can be reactivated by supplying an amount of current such that the cathode potential is more positive than the theoretical hydrogen evolution potential at −0.71 V vs Ag/AgCl (3 M KCl) at a pH of 8.5 under anaerobic conditions and at a pH of greater than 7.5.

The aqueous solution comprising dissolved carbon dioxide may be a solution purposely made or a natural occurring solution such as sea water. Purposely made aqueous solutions may be obtained by contacting a gas comprising carbon dioxide with an aqueous solution having a pH of above 7.5 to obtain an aqueous solution wherein a major part of the dissolved carbon dioxide is present as a bicarbonate ion and/or as a carbonate ion. The aqueous solution having a pH of above 7.5 in such an absorption process step suitably comprises sodium ions or sodium and potassium ions as described above. The carbon dioxide comprising gas may be any gas comprising carbon dioxide such as for example natural gas and especially biogas from anaerobic digestion of for example livestock manure and municipal or industrial wastewater treatment. The gas may also be syngas, associated gas, refinery offgas, amine acid gas or landfill gas. The syngas may be prepared by gasification of biomass, coal or other organic residues. The feed gas suitably comprises between 1 and 98 vol. % methane and between 2 and 60 vol. % carbon dioxide on a dry basis and preferably between 10 and 50 vol. % methane and between 30 and 40 vol. % carbon dioxide on a dry basis. Up to 97 vol. % of other compounds may be present on a dry basis. Other compounds may be hydrogen sulphide, ammonia, methane, ethane, propane, mercaptans, H₂, N₂, and/or CO.

The absorption process step is typically performed in an absorption or contacting column where gas and liquid flow counter-currently. Suitably the absorption process step is performed in a vertical column wherein continuously the carbon dioxide comprising gas is fed to the column at a lower position of the column and the aqueous alkaline solution is continuously fed to a higher position of the column such that a substantially upward flowing gaseous stream contacts a substantially downwards flowing liquid stream. The column is further provided with an outlet for the loaded aqueous solution at its lower end and an outlet for the gas having a lower content of carbon dioxide at its upper end.

The pH of the aqueous solution in the absorption process will decline as a result of the carbon dioxide which is dissolved. For this reason the pH of the starting aqueous solution and its composition should preferably be such that in the obtained liquid aqueous solution in the major part of the dissolved carbon dioxide is present as a bicarbonate ion and/or as a carbonate ion. Optionally alkaline compounds can be added after the absorption step to achieve these conditions.

The temperature in the absorption process step may be between 5 and 70° C. and preferably between 3° and 40° C. The pressure may be in the range of from 0 bara to 100 bara, preferably of from atmospheric pressure to 80 bara.

The absorption process step is preferably performed such that no oxygen is dissolved in the loaded aqueous solution. This may be achieved by starting with a carbon dioxide gas having a low oxygen content. If the gas however contains oxygen some pretreatment may be required. Traces of oxygen are allowed as traces of oxygen will also enter the cathode compartment via the membrane from the anode where oxygen is formed in one preferred embodiment.

Preferably the gas comprising carbon dioxide is counter currently contacted with an aqueous solution having a pH of above 7.5 and comprising dissolved methane as obtained in the process according to this invention and wherein the gas strips the methane from the aqueous solution to obtain a gas comprising methane. In this way methane is effectively isolated from the aqueous reaction mixture while carbon dioxide is absorbed using the same unit operation.

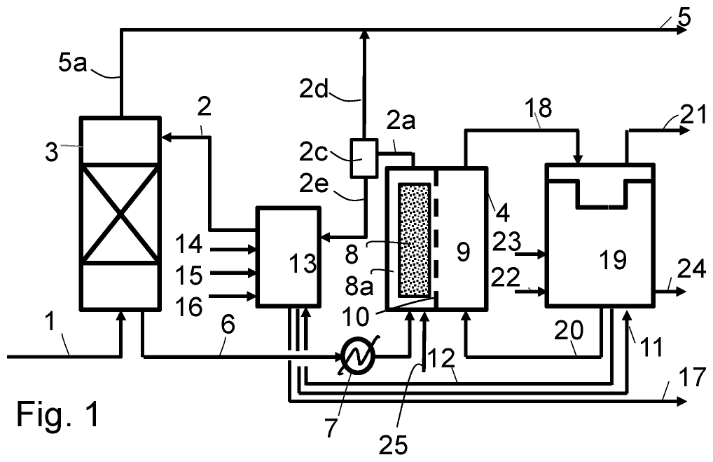

FIG. 1 shows a possible process scheme for the process of this invention. A gas comprising carbon dioxide (1) is counter currently contacted in absorption column (3) with an aqueous solution (2) having a pH of above 7.5 and comprising dissolved methane as obtained in reactor (4). In column (3) the gas (1) strips the methane from the aqueous solution (2) to obtain a gas (5a) comprising methane. Part of the methane as formed in reactor (4) is separated as a gas (2d) in knockout vessel (2c) from aqueous reaction mixture (2a) and combined with the aforementioned gas (5a) to combined gas stream (5). A liquid aqueous solution (2e) is obtained in the knock-out vessel (2c). In this way methane is effectively isolated from the aqueous reaction mixture (2,2a) while carbon dioxide is absorbed in the same column (3). The methane rich gas is obtained as gas stream (5). In the obtained aqueous solution (6) comprising dissolved carbon a major part of the dissolved carbon dioxide is present as a bicarbonate ion and/or as a carbonate ion. This aqueous solution (6) is cooled in heat exchanger (7) and fed to an electron charged packed bed (8) comprising of a carrier and a biofilm of microorganisms under anaerobic conditions. In the electron charged packed bed (8) carbon dioxide as the bicarbonate ion and/or as the carbonate ion reacts to methane. It is believed that this is achieved without the formation of hydrogen as an intermediate reaction product. The electron charged packed bed (8) is part of a biocathode (8a) in a bioelectrochemical reactor (4) further comprising an anode (9) and an ion exchange membrane (10) to avoid oxygen as may be formed at the anode (9) to flow to the biocathode (8a).

A hydrogen rich stream (25), for example a rest stream of another process, may be supplied to biocathode (8a) of bioelectrochemical reactor (4). This hydrogen will be converted with carbon dioxide to methane in the bioelectrochemical reactor (4).

The aqueous reaction mixture (2,2a) obtained at the biocathode (8a) is fed to column (3) via a mixture vessel (13). To mixture vessel (13) make up water (14), make up caustic (15) and make up nutrients and vitamins (16) may be added. A catholyte bleed stream (17) discharges part of the catholyte from the process.

At the anode water is oxidised and the oxygen as formed is discharged via (18) to an anolyte buffer vessel (19). The anolyte compartment of the reactor (9) is fed with fresh anolyte via (20). In this vessel molecular oxygen is separated as (21). Make up water (22) and make up caustic (23) is added and an anolyte bleed stream (24) discharges part of the aqueous solution from the process.

Part of the anolyte (12) is fed to the mixture vessel (13) to become part of the catholyte and part of the catholyte (11) is fed to the anolyte buffer vessel (19) to become part of the anolyte. These streams (11,12) may be treated to lower the content of oxygen and methane as described above.

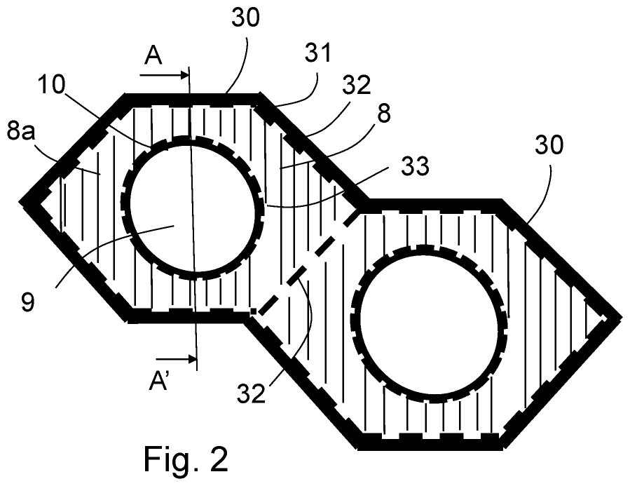

FIG. 2 shows a top view cross-sectional view of a possible bioelectrochemical reactor configuration (4). The bioelectrochemical reactor (4) is comprised of more numerous hexagonal shaped cells (30) functioning as a current collector. Each cell (30) comprises an electron charged packed bed of activated carbon granules (8) as part of a biocathode (8a), an anode (9) and an ion exchange membrane (10) to avoid oxygen as may be formed at the anode (9) to flow to the biocathode (8a) and methane as may formed at the biocathode (8a) to flow to the anode (9). The cells may advantageously be positioned next to each other as in a honeycomb as shown for two cells (30) in FIG. 2. The electron charged packed bed of activated carbon granules (8) contacts an inside current collector (32,33) made of stainless steel. Two adjoining cells (30) may share one inside current collector (32) as shown. When multiple cells are combined the current collector (32) suitably has a honeycomb structure where the cells of the honeycomb are the cells (30). The combined numerous hexagonal shaped cells (30) are further provided with an outer wall (31) as shown for the two combined cells (30). The outer wall (31) may optionally be spaced away from the honeycomb shaped current collector (32) and may have another shape.

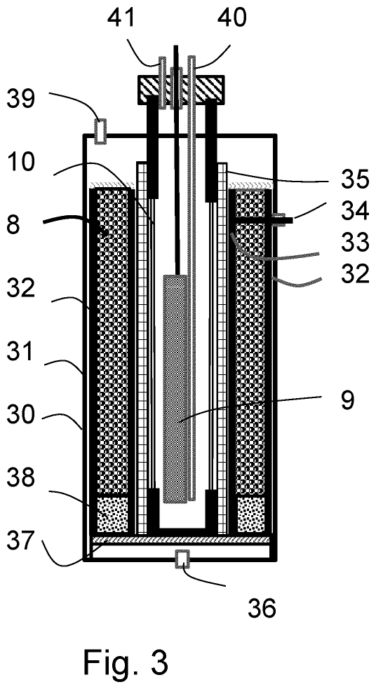

FIG. 3 shows the cross-sectional view AA′ of a single hexagonal cell of FIG. 2. The anode (9) is shown in more detail as a metal plate submerged in the anolyte. Inside current collectors (32,33) are in contact with power source (34). Between inside current collector (33) and membrane (10) a membrane protector (35) made of a PTFE mesh is present. An inlet (36) for stream (6) of FIG. 1 is present at the lower end. The liquid flows via distribution plate (37) and liquid distribution material (38) to the packed bed (8). An outlet for steam (2a) of FIG. 1 is provided at the upper end of the cell (30). Further an inlet (40) for stream (20) and an outlet (41) for stream (18) of FIG. 1 is provided at the upper end of the cell (30).

The invention is illustrated by the following non-limiting examples. In these examples the energy efficiency of the process is shown. This energy efficiency is defined as follows. In general, the energy efficiency of an electron driven process as the process according to this invention is described as the external electrical energy that ends up in the aimed end-product methane. The energy efficiency is calculated as Equation 1.

$${η}_{energy} = {η}_{product} \times {η}_{voltage} \qquad ( Eq . l )$$

For the CH₄ producing process of this invention, η_{product} is the current-to-methane efficiency. This is described as the efficiency of capturing electrons from the electric current in the form of CH₄, which is calculated as shown in Equation 2.

$${η}_{product} = \frac{{N}_{C H 4} \cdot 8 \cdot F}{{\int }_{t = 0}^{t} I \cdot dt} \qquad ( Eq . 2 )$$

Where N_{CH4} is the amount of methane produced (in mole) during a certain amount of time (t); 8 is the amount of electrons required to produce 1 molecule of CH₄; F is the Faraday constant (96485 C/mol e⁻); I is the current (A).

The voltage efficiency (η_{voltage}) is described as the part of the energy input (i.e. the required cell voltage to run the system) which ends up in CH₄, which is calculated as shown in Equation 3.

$${η}_{voltage} = \frac{- Δ {G}_{CH 4}}{{E}_{cell} \cdot 8 \cdot F} \qquad ( Eq . 3 )$$

In this equation ΔGC_{H4} is the change in Gibb's free energy of oxidation of CO₂ to CH₄ (890×10³ J/mol CH₄); Ecell is the applied cell voltage (V); 8 is the amount of electrons required to produce 1 molecule of CH₄; F is the Faraday constant (96485 C/mol e⁻).

### Example 1

A biocathode was operated at halo alkaline medium, containing 0.6 M carbonate/bicarbonate buffer with a conductivity of around 40 mS/cm (Na:K of 4:1). The medium contained 0.2 g/L NH₄Cl, 1 mL/L Wolfe's vitamin solution and 1 mL/L Wolfe's modified mineral solution. The BES setup is similar to the BES setup described in Liu, Dandan, Marta Roca-Puigros, Florian Geppert, Leire Caizán-Juanarena, Na Ayudthaya, P. Susakul, Cees Buisman, and Annemiek Ter Heijne. “Granular carbon-based electrodes as cathodes in methane-producing bioelectrochemical systems.” Frontiers in bioengineering and biotechnology 6 (2018): 78. The cathode electrode was 10.3 g of granular activated carbon, which was fully packed in the cathodic chamber. A plain graphite plate was used as a current collector. An anodic chamber and a cathodic chamber with a flow channel of 33 cm³ each (11 cm×2 cm×1.5 cm). The anodic chamber and cathodic chamber were separated by a cation exchange membrane with a projected surface area of 22 cm² (11 cm×2 cm). The catholyte and anolyte were recirculated over a catholyte and anolyte recirculation bottles. The total volume of anolyte and catholyte were 500 mL and 330 mL, respectively. In order to remove O₂ produced at the anode electrode, a high anolyte flow rate of 94 mL/min was used. Also, N₂ was continuously bubbled at the rate of 80 mL/min in the anolyte recirculation bottle. The catholyte recirculation rate was 11 mL/min.

The cathodic chamber was inoculated with 30 mL of anaerobic sludge from an upflow anaerobic sludge blanket (UASB) digestion in Eerbeek, The Netherlands. The volatile suspended solids of the inoculated anaerobic sludge was 30.6 g/L. The methane-producing BES was galvanostatically controlled (fixed current) by a potentiostat (with a current density 4 A/m²). In addition, cell voltage was manually monitored via a multimeter. Liquid samples for pH and conductivity measurements were taken twice per week for both anolyte and catholyte. After a start-up period (not shown), the following results were obtained.

In the first period (25 days), the catholyte contained trace amounts of dissolved phosphate, i.e. <0.1 mM. The coulombic efficiency in this period was between 35% and 60%. That means that the remaining electrons which were supplied to the biocathode ended up in biological growth and other end-products. It was found that acetate was one dominating by-products, which is typically formed by acetogenesis. No significant amount of H₂ was detected. To suppress this process, at day 25 phosphate levels were increased to 5 mM in the catholyte. While an initial increase in coulombic efficiency could be noticed, following days did not show improvement in the coulombic efficiency. In contrast, more supplied electrons ended up in acetate. Hence, at day 50, the phosphate concentrations were increased to 50 mM. An immediate increase in CH₄ formation was observed, bringing the coulombic efficiency towards >80%.

During the entire period of operation, the voltage efficiency did not change. This is because i) the applied potential at the biocathode always remained between −0.73 and −0.65 V, ii) the applied potential at the anode remained constant and iii) applied current density remained constant. Hence, a change in coulombic efficiency directly resulted in a change in energy efficiency. While in the period of day 0-50 the energy efficiency fluctuated between 20-50%, at higher phosphate levels the energy efficiency increased to 67%.

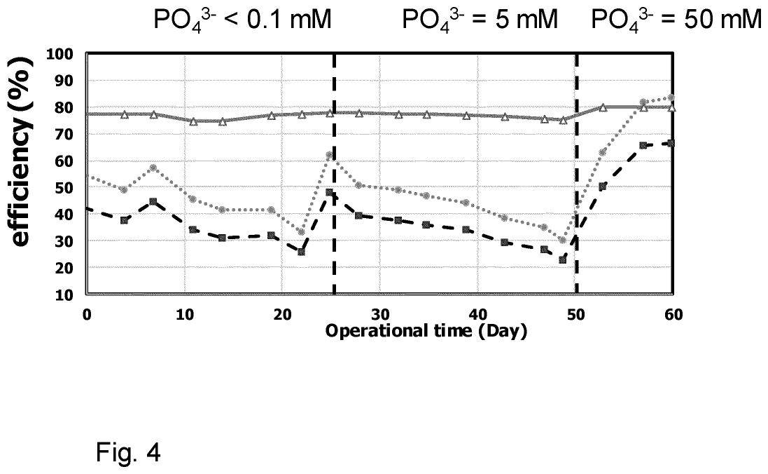

See also FIG. 4 where the current to methane efficiency is represented by the dotted line, the voltage efficiency by the solid line and the energy efficiency by the dashed line.

### Example 2

A biocathode was operated at high saline medium, containing 0.6 M carbonate/bicarbonate buffer with a conductivity of around 40 mS/cm (Na:K of 4:1). The medium contained 0.2 g/L NH₄Cl, 1 mL/L Wolfe's vitamin solution and 1 mL/L Wolfe's modified mineral solution. Additionally, the phosphate concentration was 50 mM. Similar setup and process control was applied as in Example 1.

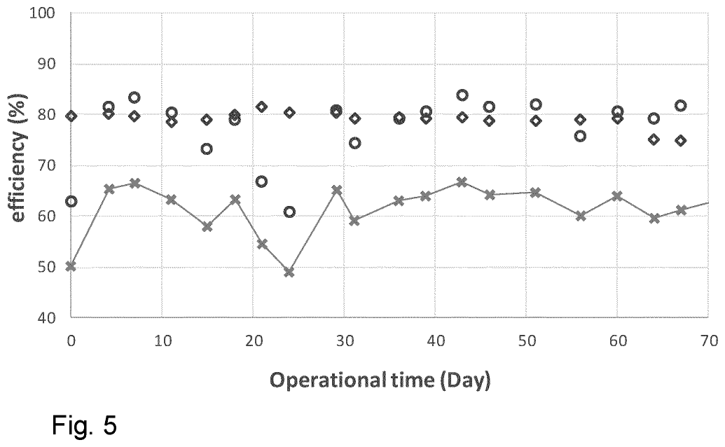

The methane-producing BES was galvanostatically controlled (fixed current) by a potentiostat (with a current density 2.5 A/m²). During a 70 days period of operation, energy efficiency was monitored over time. While maintaining the phosphate concentrations at 50 mM, also the energy efficiency over the entire period was between 55 to 67%, except for day 24 (49%). The latter might be explained as an outlier. Thus a stable process is illustrated as also shown in FIG. 5 where the current to methane efficiency is represented by the open circles, the voltage efficiency by the solid diamonds and the energy efficiency by the crosses and connecting line.

1. A process to convert carbon dioxide to methane by contacting an aqueous solution comprising dissolved carbon dioxide with an electron charged packed bed comprising of a carrier and a biofilm of microorganisms under anaerobic conditions
wherein the pH of the aqueous solution is above 7.5,
wherein the aqueous solution comprises between 0.3 and 4 M sodium cations or between 0.3 and 4 M sodium and potassium cations and
wherein the aqueous solution comprises more than 20 mM phosphate ions.

2. The process according to claim 1, wherein the aqueous solution comprises between 0.4 and 2 M sodium cations or between 0.4 and 2 M sodium and potassium cations.

3. The process according to claim 1, wherein the aqueous solution comprises between 0.5 and 1.5 M sodium cations or between 0.5 and 1.5 M sodium and potassium cations.

4. The process according to claim 1, wherein the carrier is comprised of activated carbon granules.

5. The process according to claim 1, wherein no power is supplied to the electron charged packed bed.

6. The process according to claim 5, wherein the electron charged packed bed is part of a biocathode in a bioelectrochemical system further comprising an anode, an ion exchange membrane, and a cathode;
wherein the packed bed is charged by applying a potential to the bioelectrochemical system resulting in a current between biocathode and anode for a certain time.

7. The process according to claim 1, wherein the electron charged packed bed is part of a biocathode in a bioelectrochemical system further comprising an anode; and
wherein at one moment in time the process is performed when the packed bed is charged by applying a potential to the bioelectrochemical system resulting in a current between biocathode and anode and wherein at another moment in time the process is performed when no power is supplied to the electron charged packed bed.

8. The process according to claim 6, wherein the process is performed in more than one bioelectrochemical systems, each system comprising of the biocathode and an anode;
wherein in one or more bioelectrochemical systems the process is performed while no power is supplied to the electron charged packed bed of these one or more bioelectrochemical systems; and
wherein power is supplied to the packed bed of one or more other bioelectrochemical system of the more than one bioelectrochemical systems such that these packed beds are charged with electrons while the process is not performed.

9. The process according to claim 7, wherein the process is performed for between 0.03 and 12 hours when no power is supplied to the electron charged packed.

10. The process according to claim 7, wherein the power supply generating the potential is electricity generated by solar and/or wind.

11. The process according to claim 6, wherein the packed bed is charged by applying a current density to the cathode electrode of between 2 and 200 A/m² or by applying a cathode potential to the current collector of the biocathode which is less negative than the hydrogen evolution potential.

12. The process according to claim 6, wherein the anode is a titanium mesh coated with iridium and or tantalum.

13. The process according to claim 6, wherein the power supply is generated by a chemical reaction at the anode.

14. The process according to claim 1, wherein the packed bed is a packed bed of activated carbon granules having an activated surface area of between 500 and 1500 m²/g; and
wherein the microorganisms are present as a biofilm on the surface of the activated surface area.

15. The process according to claim 1, wherein the aqueous solution comprising dissolved carbon dioxide is obtained by contacting a gas comprising carbon dioxide with an aqueous solution having a pH of above 7.5 to obtain an aqueous solution wherein a major part of the dissolved carbon dioxide is present as a bicarbonate ion and/or as a carbonate ion.

16. The process according to claim 1, wherein the microorganisms are halophilic microorganisms.
